(19) United States
(12) Patent Application Publication
(10) Pub. No.: US 2021/0299009 A1
(43) Pub. Date: Sep. 30, 2021

(54) SILICONE OIL FREE ANTIPERSPIRANTS

(71) Applicant: Henkel AG & Co. KGaA, Duesseldorf (DE)

(72) Inventors: THOMAS DOERING, Dormagen (DE); SANDRA MAUSBERG, Haan (DE)

(73) Assignee: Henkel AG & Co. KGaA, Duesseldorf (DE)

(21) Appl. No.: 17/217,020

(22) Filed: Mar. 30, 2021

(30) Foreign Application Priority Data
Mar. 30, 2020 (DE) 10 2020 108 727.6

(51) Int. Cl. A61K 8/26; A61K 8/37; A61K 8/33; A61Q 15/00

(52) U.S. Cl. CPC A61K 8/26; A61K 8/37; A61K 2800/31; A61Q 15/00; A61K 2800/34; A61K 8/33

(57) ABSTRACT

The present disclosure relates to an antiperspirant cosmetic composition which provides good efficacy without silicone oils. Furthermore, the present disclosure relates to a cosmetic method for reducing perspiration and/or body odor caused by perspiration using a composition as contemplated herein.

### CROSS-REFERENCE TO RELATED APPLICATION

This application claims priority to German Patent Application No. 10 2020 108 727.6, filed Mar. 30, 2020, which is incorporated herein by reference in its entirety.

### TECHNICAL FIELD

The present disclosure relates to an antiperspirant cosmetic composition that provides good efficacy without silicone oils. Furthermore, the present disclosure relates to a cosmetic method for reducing perspiration and/or body odor caused by perspiration using a composition as contemplated herein.

### BACKGROUND

Washing, cleaning, and caring for one's own body is a basic human need and modern industry is constantly trying to meet these needs in a variety of ways. Particularly important for daily hygiene is the permanent elimination or at least reduction of body odor and underarm wetness. Numerous special deodorizing or antiperspirant body care products are known in the state of the art, which have been developed for use in body regions with a high density of sweat glands, especially in the armpit area. These are available in a wide variety of dosage forms, for example as powder, in stick form, as aerosol spray, pump spray, liquid and gel roll-on application, cream, gel and as impregnated flexible substrates (deodorants).

State-of-the-art cosmetic antiperspirants contain, in addition to at least one oil or wax and a fragrance component or perfume, at least one antiperspirant compound, for example in the form of aluminum salts and/or aluminum-zirconium salts, in the form of halides and/or hydroxyhalides of aluminum and/or zirconium.

Antiperspirants and deodorants usually contain silicone oils to create a good feeling on the skin. Decamethylcyclopentasiloxane is frequently used. Furthermore, corresponding silicone oils have the advantage that they are volatile so that they do not leave stains or smear films on the user's clothing. However, the volatility of silicone oils causes white stains to be visible on the user's clothing. However, silicone oils are increasingly criticized because of their impact on the environment.

There is therefore a need for antiperspirants and deodorants that do not pollute the environment but at the same time have a good performance in terms of sweat reduction or reduction of body odor caused by sweat. In addition, the application of the agent to the skin should not stain the user's clothing.

### BRIEF SUMMARY

This disclosure provides an anhydrous antiperspirant cosmetic composition comprising:
a) from about 5 to about 40% by weight of at least one activated aluminum sesquichlorohydrate salt,
(b) from about 7 to about 30% by weight of at least one alkyl triester of a C4-8 tricarboxylic acid with a C1-4 alkyl alcohol,
c) from about 7 to about 30% by weight of at least one non-volatile ester and
d) from about 7 to about 30% by weight of PPG-14 butyl ether, the composition being free from volatile silicone oils.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first object of the present disclosure is therefore an anhydrous cosmetic composition comprising:
a) about 5 to about 40% by weight of at least one aluminum sesquichlorohydrate salt, preferably of the general formula Al₂OH₆₋ₓClₓ, wherein x is about 1.05 to about 1.59,
(b) about 7 to about 30% by weight of at least one alkyl triester of a C4-8 tricarboxylic acid with a C1-4 alkyl alcohol,
(c) about 7 to about 30% by weight of at least one non-volatile ester and
(d) 7 about to about 30% by weight of PPG-14 butyl ether, the composition being free from volatile silicone oils.

Surprisingly, it was found that an aluminum salt can be suspended in an oil mixture without volatile silicone oils and especially without any silicone oils, thus obtaining a highly effective agent. The oil blend contains at least one alkyl triester of a C4-8 tricarboxylic acid with a C1-4 alkyl alcohol, PPG-14 butyl ether and non-volatile esters.

Preferably, the composition is free of silicone oils, that is, free of volatile and non-volatile silicone oils. The cosmetic composition is an anhydrous suspension.

Antiperspirant compositions are often offered in dosage forms that feature metal. The presence of water in these cases can lead to corrosion of the dosage form. Therefore, the composition as contemplated herein is anhydrous. Anhydrous in the sense of the present disclosure means that the water content is less than about 0.5% by weight, preferably about 0.2% by weight or less, about 0.1% by weight or less.

Particularly preferably, no free water is present in the composition. This can be presented, for example, as an aerosol spray, roll-on application, or deodorant cloth (impregnated flexible substrate).

The composition is free of volatile silicone oils, preferably it is free of any silicone oils. In terms of the present disclosure , this means that the amount of volatile silicone oils or, in a preferred embodiment, any silicone oils does not exceed about 0.1% by weight. The amount of volatile silicone oils or, in a preferred embodiment, any silicone oils is thus in the range of about 0 to about 0.1% by weight. If the cosmetic composition as contemplated herein contains volatile silicone oils or, in a preferred embodiment, volatile or non-volatile silicone oils, these have not been actively added to the composition but are present as an impurity of one of the metered components. Corresponding contamination cannot always be completely avoided.

The term “volatile silicone oils” refers to compounds which, at about 20° C. and an ambient pressure of about 1,013 hPa, have a vapor pressure of about 2.66 Pa to about 40,000 Pa, preferably of about 10 to about 12,000 hPa, more preferably of about 13 to about 3,000 hPa, of about 15 to about 500 hPa sharp edges.

As contemplated herein, the term “antiperspirant” is understood to mean the decrease or reduction of perspiration of the body's sweat glands or the reduction of body odor caused by sweat. Thus, an antiperspirant cosmetic composition within the meaning of the present disclosure may be an antiperspirant or a deodorant. Preferably, it is an antiperspirant. Where quantities are given in this application, these are percentages by weight, unless explicitly stated otherwise. Unless otherwise stated, all quantities refer to the total weight of the composition as contemplated herein. Any blowing agents that may have been added do not count as part of the composition as contemplated herein, so all quantities given refer to the total weight of the blowing agent-free composition, unless otherwise stated.

The composition as contemplated herein has at least one aluminum salt known as aluminum sesquichlorohydrate. The use of activated aluminum sesquichlorohydrate in combination with the oil mixture as contemplated herein has proven to be particularly advantageous in terms of antiperspirant performance Preferred antiperspirant cosmetic compositions are exemplified in that they contain at least one aluminum sesquichlorohydrate salt of formula Al₂OH₆₋ₓClₓ, wherein x is about 1.05 to about 1.59 (i.e. a molar ratio of aluminum to chloride of from about 1.26:1 to about 1.90:1), preferably x is about 1.06 to about 1.48 (i.e. a molar ratio of aluminum to chloride of from about 1.35:1 to about 1.88:1), more preferably x is about 1.08 to about 1.42 (i.e. a molar ratio of aluminum to chloride of from about 1.40:1 to about 1.88:1), in particular x is about 1.08 to about 1.21 (i.e. a molar ratio of aluminum to chloride of from about 1.65:1 to about 1.88:1). The use of such aluminum salts with the previously stated molar ratios of aluminum to chloride has been shown to be particularly beneficial in terms of antiperspirant activity, reduced residue formation, and improved dryness sensation. Furthermore, it may be preferred as contemplated herein if the aluminum salts used have a high chloride content. Preferred antiperspirant cosmetic compositions are therefore exemplified in that they contain at least one aluminum salt with a chloride content of from about 5% to about 35% by weight, preferably from about 10% to about 30% by weight, more preferably from about 15% to about 25% by weight, from about 17% to about 20% by weight, based on the total weight of the aluminum salt. The use of such salts leads to a high antiperspirant effect as well as to a reduced formation of residues compared to aluminum salts with different, especially lower, chloride contents. The chloride content of the aluminum salts can be determined, for example, by potentiometric titration against a silver nitrate solution as standard.

It has been found advantageous in the context of the present disclosure if the antiperspirant cosmetic composition contains aluminum sesquichlorohydrate salts, particularly those of the formula Al₂OH₆₋ₓClₓ, wherein x is about 1.05 to about 1.59, which are present in activated form and have a high proportion of polymeric aluminum complexes (hereinafter also referred to as Al complexes) with a low molecular weight. These polymeric Al complexes are formed by dissociation of aluminum salts into Al(III) aqua complexes and subsequent formation of polymeric Al complexes with hydroxide and oxide bridges between aluminum ions. However, polymeric Al complexes with a high molecular weight exhibit a lower antiperspirant effect than polymeric Al complexes with a low molecular weight. For example, the formation of polymeric Al complexes with low molecular weights can be determined by size exclusion chromatography. For this purpose, a column containing silanized porous silica microspheres with a particle size of 5 μm is used (for example, Agilent Zorbax PSM 60-S column, which is commercially available from Agilent). 0.02 M HCl is suitable as eluent. Upon analysis of the aluminum salts used as contemplated herein, 5 to 6 peaks are obtained, representing polymeric Al complexes with different molecular weight ranges. Peaks 1 and 2 represent polymeric Al complexes with an average molecular weight of about 5,000 to about 10,000 g/mol, peak 3 represents polymeric Al complexes with an average molecular weight of about 2,000 to about 4,000 g/mol, and peak 4 represents polymeric Al complexes with an average molecular weight of about 500 to about 1,500 g/mol. Finally, peaks 5 and 6, respectively, represent polymeric Al complexes with average molecular weights ranging from about 135 to about 270 g/mol.

The area of the respective peak is directly proportional to the amount of polymeric Al complexes of the respective molecular weight range. Therefore, by comparing the area of one peak with the total area of all peaks obtained, the proportion of polymeric Al complexes of the corresponding molecular weight range in the total amount of polymeric Al complexes formed in the aluminum salt used as contemplated herein can be directly determined.

Peak 4 contains the polymeric Al complexes of low molecular weight that are significant for the antiperspirant effect. About the antiperspirant effect, it is therefore advantageous as contemplated herein if the area fraction of peak area 4 lies within a certain range compared to the total area of all peak areas 1 to 5 or 1 to 6 in the chromatogram. Preferred antiperspirant cosmetic compositions as contemplated herein are therefore exemplified in that they contain at least one aluminum sesquichlorohydrate salt, preferably of the formula Al₂OH₆₋ₓClₓ, wherein x is about 1.05 to about 1.59with a peak area fraction of peak 4 to the total area of all peaks in the chromatogram of from about 40 to about 80%, preferably from about 42 to about 75%, more preferably from about 45 to about 70%, from about 50 to about 65%. The peak area fraction of peak 4 with respect to the total area of all peaks in the chromatogram was determined by size exclusion chromatography using an Agilent Zorbax PSM 60S column with 0.02 M HCl as eluent. The total area of all peaks here refers to the sum of all peaks obtained for the aluminum salt in the chromatogram. The use of such aluminum salts has proven to be particularly beneficial in terms of reduced residue formation as well as improved dryness sensation.

The at least one aluminum salt, particularly those of the formula Al₂OH₆₋ₓClₓ, wherein x is about 1.05 to about 1.59, is preferably used in certain ranges of amounts. Therefore, as contemplated herein, it is advantageous if the cosmetic composition contains—based on its total weight—from about 8 to about 38% by weight, preferably from about 10 to about 35% by weight, preferably from about 12 to about 35% by weight, in particular from about 15 to about 30% by weight, of at least one aluminum salt as described above. The use of the total amounts of aluminum salt indicated above results in excellent antiperspirant performance without causing incompatibilities with other ingredients of the cosmetic composition as contemplated herein or excessive residue formation on textiles.

As a second essential ingredient b), the antiperspirant cosmetic composition as contemplated herein contains at least one alkyl triester of a C4-8 tricarboxylic acid with a C1-4 alkyl alcohol. In particular, the tricarboxylic acid is selected from citric acid, aconitic acid, isocitric acid and oxalsuccinic acid, preferably it is selected from citric acid and isocitric acid. The C1-4 alkyl alcohol is particularly selected from ethanol and propanol, especially preferably propanol. Thus, triethyl or tripropyl esters of citric acid or of isocitric acid are particularly preferred. Triethyl citrate is particularly preferred. Preferred antiperspirant cosmetic compositions as contemplated herein are exemplified in that they comprise—based on their total weight—from about 7 to about 25% by weight, preferably from about 7 to about 20% by weight, more preferably from about 7.0 to about 16% by weight, from about 7.0 to about 12% by weight of at least one alkyl triester of a C4-8 tricarboxylic acid with a C1-4 alkyl alcohol.

As a third essential ingredient c), the antiperspirant cosmetic composition as contemplated herein contains at least one non-volatile ester. This is different from the triester (component b)). In the context of the present disclosure, it is preferred if the at least one non-volatile ester has a certain refractive index Rf. Preferred antiperspirant cosmetic compositions as contemplated herein are therefore exemplified in that they contain at least one non-volatile ester having a refractive index Rf, measured at 20° C., of from about 1.481 to about 1.590, preferably from about 1.481 to about 1.560, more preferably from about 1.481 to about 1.540, from about 1.481 to about 1.500. The use of at least one non-volatile ester with a refractive index Rf in the range indicated above has been shown to be particularly advantageous in terms of reduced residue formation. The refractive index Rf can be determined using a refractometer, for example. Furthermore, it has been found advantageous in the context of the present disclosure if certain non-volatile esters are used. It is therefore particularly preferred in the context of the present disclosure if the cosmetic composition contains at least one non-volatile ester selected from the group including C12-C15 alkyl benzoate, phenoxyethyl caprylate, ethylhexyl palmitate, isopropyl myristate and mixtures thereof, C12-C15 alkyl benzoate and phenoxyethyl caprylate and ethylhexyl palmitate and isopropyl myristate, especially C12-C15 alkyl benzoate and phenoxyethyl caprylate. The use of the above compounds in combination with the at least one aluminum salt of the above formula, as well as that of component d) leads to an excellent antiperspirant effect as well as to a reduced residue formation of white spots.

In the context of the as contemplated herein present disclosure, it is also preferred if the at least one non-volatile ester c) is used in specific ranges of amounts. Preferred antiperspirant cosmetic compositions as contemplated herein are therefore exemplified in that they contain—based on their total weight—from about 7 to about 25% by weight, preferably from about 7 to about 20% by weight, preferably from about 7.0 to about 16% by weight, from about 7.0 to about 12% by weight, of at least one nonvolatile ester, in particular C12-C15 alkyl benzoate and/or phenoxyethyl caprylate and/or ethylhexyl palmitate and/or isopropyl myristate. The non-volatile ester C12-C15 alkyl benzoate is particularly preferred.

As the fourth essential ingredient d), the antiperspirant cosmetic composition as contemplated herein contains PPG-14 butyl ether, i.e., a polyoxypropylene (14) butyl ether. PPG-14 butyl ether is a propylene glycol ether of butyl alcohol, which can be exemplified by the following formula: C4H9(OC(CH3)CH2)nOH, where n is 14. Due to the propylene glycol content, PPG-14 butyl ether is exemplified by a moderately hydrophilic character, which is very suitable for dispersing hydrophilic ingredients of the cosmetic composition. PPG-14 butyl ether can also be used to disperse salts, such as the aluminum salt used in the disclosure, in the oil mixture. In addition, PPG-14 butyl ether makes an important contribution to the non-greasy skin feel of the oil blend as contemplated herein.

Preferred antiperspirant cosmetic compositions as contemplated herein are exemplified in that they contain—based on their total weight—from about 7% to about 25% by weight, preferably from about 7% to about 20% by weight, preferably from about 7.0% to about 16% by weight, from about 7.0% to about 12% by weight of PPG-14 butyl ether. Components b), c) and d) together form an oil mixture in which the aluminum salt used as contemplated herein is present. Surprisingly, it has been shown that each of the components of the oil mixture must be present in a proportion of at least about 7% by weight for the activated aluminum sesquichlorohydrate to be stably suspended and readily released from the oil on the skin. For this to be the case, it has been shown that an HLB requirement of the oil blend of > about 8 is beneficial. The oil mixture of components b), c) and d) therefore preferably has an HLB requirement of about 9 or more, of about 10 or more, preferably of about 11 or more. The HLB (Hydrophilic Lipophilic Balance) requirement of the oil blend is the sum of the weighted HLB requirement of the individual oils. At an HLB requirement <8, the oil matrix is too hydrophobic, and the aluminum salt can no longer be released efficiently on the skin.

If the oil mixture has the individual components b), c) and d) in a proportion of less than about 7% by weight, an effective, stable composition cannot be obtained. The upper limits of the ingredients are determined by their miscibility and compatibility with each other and with other ingredients of the composition.

If the composition as contemplated herein is present as an aerosol spray, it further comprises at least one propellant. In the context of the present disclosure, it is preferred to use certain blowing agents. Advantageous antiperspirant cosmetic compositions as contemplated herein are therefore exemplified in that they contain at least one propellant selected from the group including propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropenes and mixtures thereof, in particular propane and/or n-butane and/or iso-butane. Furthermore, as contemplated herein, it is also possible to use mixtures of the blowing agents with hydrophilic blowing agents, such as carbon dioxide and/or dimethyl ether.

To ensure sufficient spray ability and complete extraction of the compositions as contemplated herein in this embodiment, the propellant is preferably used in certain quantity ranges. It is therefore advantageous in the context of the present disclosure if the cosmetic composition contains—based on its total weight—from about 40% to about 90% by weight, preferably from about 55% to about 88% by weight, preferably from about 60% to about 86% by weight, from about 70% to about 85% by weight, of at least one propellant, in particular propane and/or n-butane and/or iso-butane. Pressure-resistant containers for such aerosols can be made of metal (aluminum, tinplate, tin), protected or non-splintering plastic, or glass coated on the outside with plastic. Pressure and breakage resistance, corrosion resistance, ease of filling, as well as aesthetic considerations, handiness, printability, etc., all play a role in their selection. Special internal protective coatings ensure corrosion resistance to the cosmetic composition of the present disclosure located in the pressure vessel. Due to the absence of water, however, the corrosiveness of the composition is significantly reduced, so that corresponding protective coatings are only required to a limited extent or not at all. In particular, the valves used have an internally painted valve head, whereby the paint and valve material are compatible with each other. If aluminum valves are used, their valve discs can be coated on the inside with Microflex paint, for example. If tinplate valves are used as contemplated herein, their valve discs can be coated on the inside with PET (polyethylene terephthalate), for example. Coating is not necessary in the compositions as contemplated herein, but it is also not troublesome. The sizes of the aerosol droplets and the respective size distribution can be adjusted for a given spray device via the quantity ratio of propellant to the other ingredients of the cosmetic composition.

However, it may be equally preferred as contemplated herein if the cosmetic composition is sprayed as a propellant-free pump spray or squeeze spray or applied as a roll-on.

The composition as contemplated herein may be in the form of a suspension, for example. To stabilize the suspension, the composition as contemplated herein may contain ingredients that can prevent or reduce sedimentation, i.e., improve the stability of the suspension. One, two, three or more suitable thickeners can be added for this purpose.

Depending on the desired application and form of application, the composition as contemplated herein may comprise further components, in particular excipients. These are preferably those selected from the group of solvents, perfume oils, emulsifiers, solubilizers, cosmetic oils, consistency agents, propellants, pH-adjusting agents, surfactants, chelating agents, skin-cooling active ingredients, skin-caring active ingredients, waxes, preservatives, plant extracts, absorbers, and mixtures of these excipients. The composition as contemplated herein not only has an antiperspirant effect but can also be used to mask odors. To this end, the composition preferably further comprises at least one fragrance. In the context of the present disclosure , “perfume” means substances with a molecular weight of about 74 to about 300 g/mol, which contain at least one osmophoric group in the molecule and have an odor and/or taste, i.e., which can excite the receptors of the hair cells of the olfactory system. Osmophoric groups are groups covalently bound to the molecular skeleton in the form of hydroxy groups, formyl groups, oxo groups, alkoxycarbonyl groups, nitrile groups, azide groups, etc. In the context of the present disclosure, perfume oils, perfumes or perfume oil components which are liquid at about 20° C. and about 1013 hPa are understood. In this context, the fragrance is preferably present in the composition as contemplated herein in an amount of about 0.001% by weight to about 10% by weight, particularly about 0.005% by weight to about 9% by weight, further preferably about 0.1% by weight to about 7% by weight, particularly preferably about 0.2% by weight to about 6% by weight and especially about 0.2% by weight to about 2% by weight, in each case based on the total weight of the composition as contemplated herein. To further support the odor-reducing effect of the composition as contemplated herein, in a preferred embodiment the composition as contemplated herein further comprises one or more deodorant active ingredients. As contemplated herein, the use of certain deodorant active ingredients has proven to be particularly advantageous. It is therefore preferred as contemplated herein if the at least one deodorant active ingredient is selected from the group of (i) arylsulfatase inhibitors, β-glucuronidase inhibitors, aminoacylase inhibitors, esterase inhibitors, lipase inhibitors, lipoxigenase inhibitors and cystathionine β-lyase inhibitors; (ii) α-monoalkylglycerol ethers with a branched or linear saturated or unsaturated, optionally hydroxylated C6-C22 alkyl radical, in particular α-(2-ethylhexyl)glycerol ethers, (iii) alcohols, in particular phenoxyethanol, benzylheptanol, 1,2-hexanediol, 1,2-octanediol, 1,2-decanediol, tropolone and butyloctanoic acid; (iv) germ-inhibiting perfume oils; (v) prebiotically active components; (vi) active ingredients that reduce the number of or inhibit the growth of skin germs from the group of staphylococci, coryne bacilli, anaerococci and micrococci that are involved in odor formation; (vii) active ingredients that reduce the number of or inhibit the growth of skin germs from the group of staphylococci, coryne bacilli, anaerococci and micrococci. (vii) Zinc and silver compounds, in particular zinc phenolsulfonate, zinc ricinoleate, bischloride(-1)-octahydroxy-pentazinc, zinc citrate and silver lactate; (viii) Sorbitan esters and lactones, in particular sorbitan caprylate and glucono-1,5-lactone; (ix) organohalogen compounds, especially triclosan, chlorhexidine and chlorhexidine gluconate; (x) benzalkonium halides, especially benzalkonium chloride and benzethonium chloride; (xi) quaternary ammonium compounds, especially cetylpyridinium chloride; (xii) antimicrobial carbonates, phosphates and sulfates, especially sodium bicarbonate, cocamidopropyl PG-dimonium chloride phosphates, disodium pyrophosphate and soya morpholinium ethosulphates; (xiii) lantibiotics; (xiv) aluminum and aluminum zirconium salts; (xv) bispyridines, especially octenidine; (xvi) antimicrobially active acids, in particular caprylic hydroxamic acid, carnesolic acid, tartaric acid and 2-butyloctanoic acid; (xvii) polyglycerol esters, in particular polyglyceryl-3 caprylates; (xviii) and mixtures thereof, in particular phenoxyethanol and/or caprylylglycol and/or 1,2-hexanediol and/or α-(2-ethylhexyl)glycerol ether and/or 2-butyloctanoic acid. The use of these active ingredients leads not only to a high and long-lasting perfuming and storage stability, but also to a high and long-lasting deodorizing effect.

Another object of the present disclosure is to use the composition as contemplated herein to prevent and/or reduce body odor and/or body perspiration.

A still further object of the present disclosure is a non-therapeutic cosmetic method for preventing and/or reducing body odor and/or perspiration of the body, wherein a cosmetic composition as previously described is applied to the skin, to the skin of the armpits, and is left on the skin of the armpits for at least about 1 hour, preferably for at least about 2 hours, preferably for at least about 4 hours, for at least about 6 hours.

Preferred embodiments of the present disclosure may be combined in any way, regardless of the embodiment or subject matter of the disclosure. Thus, preferred embodiments described for the composition apply correspondingly to the use and the method.

### EXAMPLES

In the following embodiment example, the present disclosure is explained in a non-limiting manner

4 compositions were prepared, one of which has the oil mixture as contemplated herein (E: as contemplated herein) and 3 other compositions (V: comparison tests).
The data are given in % by weight.

| | E | V1 | V2 | V3 |
|---|---|---|---|---|
| Activated aluminum | 33.3 | 33.3 | 33.3 | 33.3 |
| sesquichlorohydrate (AASCH 3143) | | | | |
| Cyclopentasiloxane | — | 43.0 | — | — |
| Dodecane | — | — | 10.0 | — |
| Polydimethylsiloxane | — | — | 10.0 | — |
| PPG14 Butyl ether | 10.0 | — | — | — |
| C₁₂-C₁₅ alkyl benzoate | 10.0 | — | 5.0 | — |
| Ethylhexyl palmitate | 26.6 | 7.0 | 21.0 | 50.1 |
| Isopropyl myristate | — | 6.6 | 6.5 | 6.5 |
| Triethyl citrate | 10.0 | — | — | — |
| Thickener | 2.5 | 2.5 | 2.5 | 2.5 |
| Propylene carbonate | 0.9 | 0.9 | 5.0 | 0.9 |
| Perfume | 6.7 | 6.7 | 6.7 | 6.7 |

The formulations were each prepared in a weight ratio of 1:5.7 filled with propellant (propane/butane 15/85) in aerosol cans. The antiperspirant effect was tested on 30 test subjects. The products were applied in one axilla for three consecutive days and the amount of sweat was determined gravimetrically 48 h after the last application in the sweat room (40 min, 38° C., 35% relative humidity) compared to the untreated axilla. The application of the products in the left or right axilla was randomized in the subject collective.

The results are shown in the following table:

| | E | V1 | V2 | V3 |
|---|---|---|---|---|
| Sweat reduction after | 17.4 | 15.0 | 5.0 | 8.0 |
| 48 h [%] | | | | |

Composition E as contemplated herein shows a significant reduction of the sweat comparable to the silicone oil containing compositions (V1). Replacing the volatile silicone oil (cyclopentsiloxane) with non-volatile ester oils (ethylhexyl palmitate) did not effectively reduce perspiration. The sweat reduction in the comparative tests V2 and V3 was not significant.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. Anhydrous antiperspirant cosmetic composition comprising:
a) from about 5 to about 40% by weight of at least one activated aluminum sesquichlorohydrate salt,
(b) from about 7 to about 30% by weight of at least one alkyl triester of a C4-8 tricarboxylic acid with a C1-4 alkyl alcohol,
c) from about 7 to about 30% by weight of at least one non-volatile ester, and
d) from about 7 to about 30% by weight of PPG-14 butyl ether, the composition being free from volatile silicone oils.

2. Composition according to claim 1, wherein it comprises the at least one alkyl triester of a C4-8 tricarboxylic acid with a C1-4 alkyl alcohol and/or the at least one non-volatile ester and/or PPG-14 butyl ether in an amount of from about 7% by weight to about 25% by weight.

3. Composition according to claim 1, comprising silicone oil as impurity in an amount proportion of about 0.5% by weight or less.

4. Composition according to claim 1, wherein an oil mixture of components b), c) and d) has an HLB requirement of about 9 or more.

5. The composition of claim 1, wherein the at least one aluminum sesquichlorohydrate salt has the general formula Al₂OH₆₋ₓClₓ, wherein x is about 1.05 to about 1.59.

6. The composition according to claim 1, wherein, for the at least one aluminum salt, a peak 4 fraction of a size exclusion chromatography is from about 40% to about 80%.

7. A composition according to claim 1, wherein the at least one alkyl triester of a C₄₋₈ tricarboxylic acid with a C₁₋₄ alkyl alcohol is chosen from triethyl or tripropyl esters of citric acid and isocitric acid, respectively.

8. Composition according to claim 1, wherein the at least one non-volatile ester is chosen from C₁₂₋₁₅ alkyl benzoate, phenoxyethyl caprylate, ethylhexyl palmitate, isopropyl myristate and mixtures thereof, C12-C15 alkyl benzoate and phenoxyethyl caprylate and ethylhexyl palmitate and isopropyl myristate.

9. Non-therapeutic cosmetic process for preventing and/or reducing body odor and/or perspiration of the body, in which an anhydrous antiperspirant cosmetic composition according to claim 1 is applied to skin of the axillae and remains on the skin of the axillae for at least about 1 hour.

10. Composition according to claim 1, comprising the at least one alkyl triester of a C4-8 tricarboxylic acid with a C1-4 alkyl alcohol and/or the at least one non-volatile ester and/or PPG-14 butyl ether in an amount of from about 7% by weight to about 20% by weight.

11. Composition according to claim 1, comprising the at least one alkyl triester of a C4-8 tricarboxylic acid with a C1-4 alkyl alcohol and/or the at least one non-volatile ester and/or PPG-14 butyl ether in an amount of from about 7% by weight to about 16% by weight.

12. Composition according to claim 1, comprising the at least one alkyl triester of a C4-8 tricarboxylic acid with a C1-4 alkyl alcohol and/or the at least one non-volatile ester and/or PPG-14 butyl ether in an amount of from about 7% by weight to about 12% by weight.

13. Composition according to claim 1, comprising silicone oil as impurity in an amount proportion of about 0 to about 0.1% by weight.

14. Composition according to claim 1, wherein an oil mixture of components b), c) and d) has an HLB requirement of about 11 or more.

15. Composition according to claim 1, wherein, for the at least one aluminum salt, a peak 4 fraction of a size exclusion chromatography is from about 42% to about 75%.

16. Composition according to claim 1, wherein, for the at least one aluminum salt, a peak 4 fraction of a size exclusion chromatography is from about 50% to about 65%.

17. Composition according to claim 1, wherein the at least one alkyl triester of a C₄₋₈ tricarboxylic acid with a C₁₋₄ alkyl alcohol triethyl citrate.

18. Composition according to claim 1, wherein the at least one non-volatile ester is chosen from C₁₂-C₁₅ alkyl benzoate and phenoxyethyl caprylate.

19. Composition according to claim 1, comprising the at least one alkyl triester of a C4-8 tricarboxylic acid with a C1-4 alkyl alcohol and/or the at least one non-volatile ester and/or PPG-14 butyl ether in an amount of from about 7% by weight to about 12% by weight, comprising silicone oil as impurity in an amount proportion of about 0 to about 0.1% by weight, wherein an oil mixture of components b), c) and d) has an HLB requirement of about 11 or more, wherein, for the at least one aluminum salt, a peak 4 fraction of a size exclusion chromatography is from about 50% to about 65%, wherein the at least one alkyl triester of a C₄₋₈ tricarboxylic acid with a C₁₋₄ alcohol triethyl citrate, and wherein the at least one non-volatile ester is chosen from C₁₂-C₁₅ alkyl benzoate and phenoxyethyl caprylate.
